(19) United States
(12) Patent Application Publication
(10) Pub. No.: US 2024/0408162 A1
(43) Pub. Date: Dec. 12, 2024

(54) Use of Extract from Morus alba L. in the Preparation of a Medicament for Preventing and/or Treating a Hepatobiliary Disease

(71) Applicants: BEIJING WEHAND-BIO PHARMACEUTICAL CO., LTD, Beijing (CN); GUANGXI WEHAND-BIO PHARMACEUTICAL CO., LTD, Hechi (CN)

(72) Inventors: Yuling LIU, Beijing (CN); Yanmin CHEN, Beijing (CN); Zhihua LIU, Beijing (CN); Xiangyang ZHU, Hechi (CN); Tingting WANG, Beijing (CN); Yuanyuan LIU, Beijing (CN); Chunfang LIAN, Beijing (CN); Qianwen SUN, Beijing (CN); Lili GAO, Beijing (CN); Yuanyuan ZOU, Beijing (CN); Hongzhen YANG, Beijing (CN); Yiqun JIN, Beijing (CN)

(21) Appl. No.: 18/804,322

(22) Filed: Aug. 14, 2024

Related U.S. Application Data
(63) Continuation of application No. PCT/CN2023/077255, filed on Feb. 20, 2023.

(30) Foreign Application Priority Data
Dec. 6, 2022 (CN) 202211553942.3

(51) Int. Cl. A61K 36/605; A61P 1/16

(52) U.S. Cl. CPC A61K 36/605; A61P 1/16; A61K 2236/39; A61K 2236/50

(57) ABSTRACT

The present invention discloses applications of extracts from Morus alba L. Disclosed by the present invention is a use of extracts from Morus alba L. in the preparation of a product for alleviating, preventing and/or treating a hepatobiliary disease. The present invention demonstrated experimentally that extracts from Morus alba L. have effects of reducing hepatic lipid content and alleviating hepatic fibrosis in NAFLD mice gavaged with extracts from Morus alba L., thereby alleviating high-fat diet-induced fatty liver without toxic and side effects on liver and kidney. The drug of the present invention exerts multi-target pharmacological effects through multi-components, and specifically can regulate the hepatic lipid content by regulating the synthesis and oxidation of fatty acids, and can also affect hepatic fibrosis, which is more conducive to the treatment of a non-alcoholic fatty liver disease.

### RELATED APPLICATIONS

The present application is a Continuation of International Application Number PCT/CN2023/077255 filed Feb. 20, 2023, which claims priority to Chinese Application Number 202211553942.3 filed Dec. 6, 2022, the disclosures of which are hereby incorporated by reference herein in their entireties.

### TECHNICAL FIELD

The present invention belongs to the field of pharmaceutical technology, in particular to a use of extracts from Morus alba L. in the preparation of a medicament for preventing and/or treating a hepatobiliary disease.

### BACKGROUND

Non-alcoholic fatty liver disease (NAFLD) is one of the most common chronic liver diseases, with an incidence of up to 25% worldwide. There are several stages such as steatosis, non-alcoholic steatohepatitis, fibrosis, and cirrhosis occurred in livers of NAFLD patients, and the risk of hepatocellular cancer is also increased by NAFLD. NAFLD includes non-alcoholic fatty liver disease (NAFL) and non-alcoholic steatohepatitis (NASH), non-alcoholic steatohepatitis (NASH is an important stage during the transformation of hepatic steatosis into fibrosis, exhibiting inflammatory cells infiltration and hepatocyte balloon-like degeneration. If this stage is not treated promptly and effectively, it will be difficult to reverse the development of hepatic sclerosis. Non-alcoholic fatty hepatic fibrosis is caused by an imbalance in the generation and degradation of the extracellular matrix (ECM) with changes in both quantity and quality mainly composed of from a type IV collagen to a fibrous collagen (such as type I and type III collagen) during a chronic liver injury. Hepatic stellate cells (HSCs) activated during the fibrosis can involve in the formation of hepatic fibrosis and the reconstruction of intrahepatic structures through proliferation and secretion of the extracellular matrix, which is also a central part in the hepatic fibrosis. Due to the complex pathogenesis of NAFLD which involves abnormal lipid metabolism, oxidative stress, mitochondrial dysfunction, intestinal ecological disorder, endoplasmic reticulum stress and the like, so far, no medicaments have been approved for non-alcoholic fatty liver disease treatment, with the most among them ongoing in the clinical phase. Non-alcoholic fatty liver results from a series of complex multi-factors, many drugs acting on a single target have unsatisfactory efficacies. Since multi-target drugs block the progression of the disease by acting on different stages in the development of non-alcoholic fatty liver disease, it becomes a new direction for investigating non-alcoholic fatty liver drugs.

As a natural medicine, Morus alba L. contains various active ingredients, such as alkaloids, flavonoids, amino acids and polysaccharides, and it thus has multiple targets.

The gallbladder, located behind the liver, has a primary role of concentrating and storing bile. Due to its elongated blind pouch structure, the gallbladder is prone to obstruct, which can cause inflammation. Cholecystitis is a common disease of digestive system with a high incidence, and women between 30 and 50 years old are the susceptible population.

Different strategies are adopted by traditional Chinese and Western medicine for the treatment of cholecystitis. Traditional Chinese medicine focuses on choleresis and improving symptoms. However, Western medicine focuses on pain relief, infection control or surgery.

### DISCLOSURE OF THE INVENTION

The object of the present invention is to provide a new use of extracts from Morus alba L. or main active ingredients thereof in a medication.

New use of extracts from Morus alba L. or main active ingredients thereof provided by the present invention is set forth in (a1) and/or (a2) below:
(a1) use of the extracts from Morus alba L. in the preparation of a product for alleviating, preventing and/or treating a hepatobiliary disease;
(a2) use of the extracts from Morus alba L. in alleviating, preventing and/or treating a hepatobiliary disease.

The hepatobiliary disease is a non-alcoholic fatty liver disease and/or cholecystitis.

The alleviating, preventing and/or treating the non-alcoholic fatty liver are shown in at least one of the followings:
1) reduction of the hepatic lipid content;
2) alleviation or inhibition of hepatic fibrosis;
3) alleviation of an increase of ALT caused by the non-alcoholic fatty liver;
4) inhibition of an increase in total cholesterol, triglyceride, and/or LDL in the serum or the liver resulted from a non-alcoholic fatty liver disease or a non-alcoholic steatohepatitis;
5) inhibition of an increase of CRP in the serum caused by a non-alcoholic fatty liver disease;
6) inhibition of the expression of genes associated with hepatic fibrosis;
7) promotion of adiponectin secretion in adipocytes;
8) inhibition of fatty acid synthesis, promotion of fatty acid oxidation;
9) inhibition of an increase of liver index caused by a non-alcoholic fatty liver disease or a non-alcoholic steatohepatitis;
10) improvement of liver NAS score for non-alcoholic steatohepatitis;
11) improvement of hepatic steatosis, balloon-like degeneration, and/or lobular inflammation caused by a nonalcoholic steatohepatitis.

The present invention also claims applications of extracts from Morus alba L. or main active ingredients thereof, as set forth in at least one of (b1) to (b16) below:
(b1) preparation of a product for reducing the hepatic lipid content in a non-alcoholic fatty liver;
(b2) preparation of a product for alleviating or inhibiting hepatic fibrosis in a non-alcoholic fatty liver;
(b3) preparation of a product for alleviating an increase of ALT caused by a non-alcoholic fatty liver;
(b4) preparation of a product for inhibiting of an increase of total cholesterol, triglyceride, total cholesterol, and/or LDL in the serum or a liver caused by a non-alcoholic fatty liver disease;
(b5) preparation of a product for inhibiting an increase of CRP in the serum in a non-alcoholic fatty liver;
(b6) preparation of a product for inhibiting the expression of genes associated with hepatic fibrosis in a non-alcoholic fatty liver;
(b7) preparation of a product for improving liver NAS score in nonalcoholic steatohepatitis;
(b8) preparation of a product for improving hepatic steatosis, balloon-like degeneration, and/or lobular inflammation in nonalcoholic steatohepatitis;
(b9) use in reducing the hepatic lipid content in a non-alcoholic fatty liver;
(b10) use in alleviating or inhibiting hepatic fibrosis in a non-alcoholic fatty liver;
(b11) use in alleviating an increase of ALT caused by a non-alcoholic fatty liver;
(b12) use in inhibiting an increase of total cholesterol, triglyceride, total cholesterol, and/or LDL in the serum or liver caused by a non-alcoholic fatty liver;
(b13) use in inhibiting an increase of CRP in the serum in a non-alcoholic fatty liver;
(b14) use in inhibiting the expression of genes associated with hepatic fibrosis in a non-alcoholic fatty liver;
(b15) use in improving liver NAS score in a nonalcoholic steatohepatitis;
(b16) use in improving heptaic steatosis, balloon-like degeneration, and/or lobular inflammation of a liver in a nonalcoholic steatohepatitis.
the product is a drug or a pharmaceutical formulation.

The non-alcoholic fatty liver (NAFLD) comprises any one of steatosis, non-alcoholic steatohepatitis (NASH), hepatic fibrosis, and hepatic cirrhosis.

The cholecystitis may be chronic or acute.

The extracts from Morus alba L. are extracts from Ramulus Mori, Cortex Mori and/or Folium Mori.

Alternatively, the extracts from Morus alba L. can also be provided in the form of commercially available Ramulus Mori total alkaloid tablet (approval number Z20200002).

The extracts from Morus alba L can be prepared according to the method documented in CN 113143997 A, specifically comprising the steps of:
1) preparing crude extracted solution from Moraceae plants;
2) subjecting the crude extracted solution to cation resin and/or optional anion resin for separation to obtain the extracts from Morus alba L.

The method can further comprise the steps of:
3) subjecting resin effluent from step 2) to alcohol precipitation and collecting the supernatant;
4) concentrating and drying the supernatant.

The method can further include a step of concentrating and drying the resin effluent from step 2).

The extracts from Morus alba L. are functional in human or a mammal.

The Moraceae plants can be selected from Morus atropurpurea Roxb., Morus multicaulis Perr., Morus alba L., Morus Serrata Roxb., Morus bombycis Koidz., or hybrid Morus alba L., and the hybrid Morus alba L. is preferably Yuesang 11, Guisangyou 62, or Sangteyou 2. Various parts of the plant such as leaves, roots, branches, barks, buds, stems, fruits and the like can be used.

The extracts from Morus alba L. can be alkaloid extracts from Morus alba L. In one embodiment of the present invention, the extracts from Morus alba L. comprise alkaloids, polysaccharides, flavonoids, and amino acids.

Preferably, the alkaloids comprise at least one of 1-deoxynojirimycin (DNJ), N-methyl-1-deoxynojirimycin, fagomine (FAG), 3-epi-fagomine, 1,4-dideoxy-1,4-imino-D-arabinitol (DAB), calystegine B2, calystegine C1, 2-O-(α-D-galactopyranosyl)-1-deoxynojirimycin, 6-O-(β-D-glucopyranosyl)-1-deoxynojirimycin, and 1,4-dideoxy-1,4-imino-(2-O-β-D-glucopyranosyl)-D-arabinitol.

Wherein, the DNJ has a weight percentage of no less than 30% of total alkaloids. Preferably, DNJ has a weight percentage of no less than 40% of total alkaloids. More preferably, DNJ has a weight percentage of no less than 50% of total alkaloids.

Preferably, based on the extracts from Morus alba L., each component has a content by weight of:

| | |
|---|---|
| alkaloids | 3-99%, |
| polysaccharides | 0.2-70%, |
| flavonoids | 0-10%, |
| amino acids | 0-50%, |
| other components | 0-25%; |

more preferably, based on the extracts from Morus alba L., each component has a content by weight of:

| | |
|---|---|
| alkaloids | 30-99%, |
| polysaccharides | 0.2-35%, |
| flavonoids | 0-2%, |
| amino acids | 0-30%, |
| other components | 0-20%; |

further preferably, based on the extracts from Morus alba L., each component has a content by weight of:

| | |
|---|---|
| alkaloids | 50-99%, |
| polysaccharides | 0.2-35%, |
| flavonoids | 0-2%, |
| amino acids | 0-30%, |
| other components | 0-20%; |

further preferably, based on the extracts from Morus alba L., each component has a content by weight of:

| | |
|---|---|
| alkaloids | 50-99%, |
| polysaccharides | 0.2-25%, |
| flavonoids | 0-1%, |
| amino acids | 0-20%, |
| other components | 0-20%. |

Preferably, based on the extracts from Morus alba L., each component has a content by weight of:

| | |
|---|---|
| alkaloids | 50-65%, |
| polysaccharides | 20-25%, |
| flavonoids | 0.5-1.5%, |
| amino acids | 3-20%, |
| other components | 0-10%. |

In one embodiment, the preparation of the extracts from Morus alba L. comprises the steps of: preparing crude extracted solution; optionally, subjecting to cation resin and/or anion resin for separation; optionally, subjecting resin effluent to alcohol precipitation; and optionally, concentrating and drying treatment. Preferably, the preparation of the extracts from Morus alba L. includes the steps of: step 1, preparing crude extracted solution; step 2), subjecting to cation resin and/or optional anion resin for separation; optional step 3), subjecting resin effluent from step 2) to alcohol precipitation; and optional step 4), concentrating and drying treatment.

In one embodiment, the extracts from Morus alba L. are prepared according to the steps of: crushing Ramulus Mori, Folium Mori or Cortex Mori, followed by reflux extraction under heating with water and/or alcohol solution or acid water at the amount of 3-20 times that of the raw medicinal material to obtain extracted solutions after repeating for 1-3 times, which are combined, concentrated, loaded onto a cation exchange resin, and washed with distilled water to remove the non-adsorbed impurities, followed by elution with 0.2-3N aqueous ammonia, and the eluate from which is concentrated and loaded onto anion exchange resin with collection of the non-adsorbed fractions, and then precipitating to remove impurities by addition of ethanol, and centrifuging with the supernatant concentrated under reduced pressure or spray dried or freeze-dried to obtain extracts.

In one embodiment, the extracts from Morus alba L. are prepared according to the steps of: crushing Ramulus Mori, Folium Mori or Cortex Mori, followed by reflux extraction under heating with water and/or alcohol solution or acid water at the amount of 3-20 times that of the raw medicinal material to obtain extracted solutions after repeating for 1-3 times, which are combined, concentrated, loaded onto cation exchange resin, and washed with distilled water to remove the non-adsorbed impurities, followed by elution with 0.2-3N aqueous ammonia, the eluate from which is concentrated and loaded onto anion exchange resin with collection of the non-adsorbed fractions, which are concentrated under reduced pressure or spray dried or freeze-dried to obtain extracts.

In one embodiment, the extracts from Morus alba L. are prepared according to the steps of: crushing Ramulus Mori, Folium Mori or Cortex Mori, followed by reflux extraction under heating with water and/or alcohol solution or acid water at the amount of 3-20 times that of the raw medicinal material to obtain extracted solutions after repeating for 1-3 times, which are combined, concentrated, loaded onto cation exchange resin, and washed with distilled water to remove the non-adsorbed impurities, followed by elution with 0.2-3N aqueous ammonia, the eluate from which is concentrated under reduced pressure or spray dried or freeze-dried to obtain extracts.

In one embodiment, the extracts from Morus alba L. are prepared according to the steps of: crushing Ramulus Mori, Folium Mori or Cortex Mori, followed by reflux extraction under heating with water at the amount of 3-20 times (preferably 4-15 times) that of the raw medicinal material to obtain extracted solutions after repeating for 1-3 times (preferably for 0.5-3 h each time), which are combined, concentrated, loaded onto cation exchange resin, and washed with distilled water to remove the non-adsorbed impurities, followed by elution with 0.2-3N aqueous ammonia, the eluate from which is concentrated and loaded onto anion exchange resin with collection of the non-adsorbed fractions, and then precipitating to remove impurities by addition of ethanol, and centrifuging with the supernatant concentrated under reduced pressure or spray dried or freeze-dried to obtain extracts.

Preferably, after the cation resin is loaded onto the column, the activation is achieved by sequentially eluting with acidic solution, alkaline solution, and acidic solution. Preferably, elution with alkaline solution is maintained until the pH of the eluate is 8.0-9.5, preferably 8.5-9.5; preferably, the alkaline solution is selected from the group consisting of aqueous ammonia solution, sodium hydroxide solution, potassium hydroxide solution, or sodium carbonate solution; preferably, the alkaline solution has a concentration of 0.5-4 mol/L. Preferably, elution with acidic solution is maintained until the pH of the eluate is 3.0-7.0, preferably 4.5-6.5. Preferably, the acidic solution is selected from the group consisting of hydrochloric acid solution, phosphoric acid solution, and dibasic sodium phosphate-citrate buffer. Optionally, the cation resin can also be rinsed with 3-5 times the column volume of deionized water after the final acidic solution elution. Preferably, the cation resins are model 732 strong acid styrene-based cation exchange resins, model 734 strong acid styrene-based cation exchange resins, and model D001 macroporous strong acid styrene-based cation exchange resins.

Preferably, the cation resin is fed with the plant raw material at a weight ratio of 1:2-20. Crude extracted solution from the plant is loaded onto the cation resin, followed by elution of the loaded cation resin with an elution agent, preferably at a concentration of 0.5-2.5 mol/L. Preferably, the elution agent is flowed at a rate of 5-10 BV/h.

Preferably, the anion resins are model 717 strong alkaline styrene-based anion exchange resins, model D201 macroporous strong alkaline styrene-based anion exchange resins, and model D218 macroporous strong alkaline acrylic-based anion exchange resins. Preferably, the anion resin is fed with the plant raw material at a weight ratio of 1:1-32. The liquid is collected as it flows out of the anion resin. Preferably, the collection is stopped when the volume of the collected liquid reaches 0.1-5 times the weight of the plant raw material feed.

Preferably, the ethanol for the alcohol precipitation treatment is fed with the plant raw material at a weight ratio of 1:20-300. The alcohol precipitation treatment is performed at a stirring rate of 40-500 rpm.

Preferably, the drug further comprises a pharmaceutically acceptable carrier. The carrier is an inactive ingredient that is suitable for the route or mode of administration and not toxic to human. The carrier can be a solid or liquid excipient. The solid excipient, for example, includes microcrystalline cellulose, mannitol, lactose, pre-gelatinized starch, low substituted hydroxypropyl cellulose, cross-linked polyvinylketone, sodium carboxymethyl starch, aspartame, calcium hydrogen phosphate, sodium lactate, poloxamer, sodium dodecyl sulfate, sodium carboxymethyl cellulose, gelatin, xanthan gum, polyvinylketone, starch, magnesium stearate, sodium carboxymethyl starch, and talcum powder; and the liquid excipient, for example, includes water, ethanol, syrup, and glycerol.

Preferably, the drug is an oral dosage form; and further preferably, the drug is a tablet, capsule, oral solution, oral emulsion, pill, granule, syrup and powder.

The inventor demonstrates by a pharmacological experiment that both SZ-A 25 μg/ml and DNJ 20 μg/ml can reduce triglycerides when hepatocytes are treated with SZ-A 25 μg/ml (containing 17.1 ug/ml DNJ) and DNJ 20 μg/ml, respectively, and SZ-A shows a better effect, it is thus speculated that other ingredients than DNJ in SZ-A can also play a role.

Both DNJ and SZ-A can increase the concentration of adiponectin with a better effect from SZ-A.

The hepatic lipid content is reduced and hepatic fibrosis is alleviated in NAFLD mice gavaged with extracts from Morus alba L. (SZ-A), thereby alleviating high-fat diet-induced non-alcoholic fatty liver without toxic side effects on the liver and kidney.

Results from the prevention experiment of extracts from Ramulus Mori shows that extracts from Ramulus Mori can reduce the body weight, liver weight, serum total cholesterol, serum low-density lipoprotein cholesterol, and serum ALT and AST levels in non-alcoholic fatty liver model mice, thus alleviating high-fat diet-induced non-alcoholic fatty liver.

The liver NAS score is improved, hepatic fibrosis is alleviated, as well as hepatic steatosis, balloon-like degeneration and lobular inflammation are improved by oral administration of extracts from Morus alba L. (SZ-A) in GAN feed-induced NASH model mice.

The inventor demonstrates by a pharmacological experiment that the total cholesterol in the SZ-A group is significantly reduced compared to the model group. At the base of the gallbladder, the gallbladder muscle layer in the model group is disordered, with significant mucosa hyperplasia and edema and detachment; while the above pathological changes are all improved in the SZ-A group.

The amount of extracts from Morus alba L. (SZ-A) to be administered and the dosing protocol can be determined depended on the severity of a disease, response to a disease, any treatment-related toxicity, and the age and health condition of a patient.

For example, in some embodiments, for the treatment of non-alcoholic fatty liver disease, mice are administered at a proposed single administration dose of 100-400 mg extracts from Morus alba L./kg body weight based on total alkaloids, which is equivalent to a single administration dose of 500-2000 mg extracts from Morus alba L./kg body weight based on total alkaloids for human;
the mode of administration is oral.

For example, in some embodiments, for the prevention of non-alcoholic fatty liver disease, mice are administered at a proposed single administration dose of 15-100 mg/kg based on total alkaloids, which is equivalent to a single administration dose of 75-500 mg/kg based on total alkaloids for human;
the mode of administration is oral.

For example, in some embodiments, for the treatment of non-alcoholic steatohepatitis, mice are administered at a proposed single administration dose of 100-400 mg/kg based on total alkaloids, which is equivalent to a single administration dose of 500-2000 mg/kg based on total alkaloids for human;
the mode of administration is oral.

The single-target drug is prone to develop resistance over time, while the drug of the present invention exerts multi-target pharmacological effects through multi-components, and specifically can regulate the hepatic lipid content by regulating the synthesis and oxidation of fatty acids, and can also affect hepatic fibrosis, which is more conducive to the treatment of non-alcoholic fatty liver disease.

In one embodiment of the present invention, any two or more ingredients of polysaccharides, flavonoids, and alkaloids in extracts from Morus alba L. have a synergistic effect; and in another embodiment of the present invention, any two or more ingredients of DNJ, DAB, and FAG in alkaloids have a synergistic effect.

### BRIEF DESCRIPTION OF DRAWINGS

The patent or application file contains one drawing/photograph executed in color. Copies of this patent with color drawing(s)/photograph(s) will be provided by the Office upon request and payment of the necessary fee.

FIG. 1 shows the changes of triglyceride and cholesterol levels in HepG2 hepatocytes after administration; Mean±SEM, compared with the control group, * P<0.05, ** P<0.01; compared with the control PA group, # P<0.05, ## P<0.01; FA represents fagomine.

FIG. 2 shows the effects of SZ-A, DNJ, FAG, and DAB on the secretion of adiponectin in 3T3-L1 adipocytes.

FIG. 3 shows the liver weights in each group of mice after administration; Mean±SEM, ## P<0.01 vs. the control group, * P<0.05, ** P<0.01, *** P<0.001 vs. the HFD group.

FIG. 4 shows the relative liver weights in each group after administration; ** P<0.01 vs. the HFD group.

FIG. 5 shows H&E staining images of the livers in each group of mice after administration.

FIG. 6 shows the results from the triglyceride and cholesterol assays in the livers of each group after administration; Mean±SEM, ### P<0.001 vs. the control group, * P<0.05, ** P<0.01, *** P<0.001 vs. the HFD group.

FIGS. 7A-7E shows the results from the blood biochemical tests in each group after administration; Mean±SEM, ### P<0.001 vs. the control group, * P<0.05, ** P<0.01, *** P<0.001 vs. the HFD group.

FIGS. 8A and 8B shows the results from the transcriptomics and fluorescence quantitative PCRs of liver tissues in each group after administration.

FIG. 9 shows the results from the western blot detections of p-AMPK, AMPK, p-ACC, and ACC proteins in liver tissues of each group after administration, # P<0.05, ## P<0.01 vs. the control group, * P<0.05, ** P<0.01, *** P<0.001 vs. the HFD group.

FIG. 10 shows the effects on the body weights of non-alcoholic fatty liver model mice in each of administration groups and prevention groups.

FIG. 11 shows the effects on the liver weights of non-alcoholic fatty liver model mice in each of administration groups and prevention groups.

FIGS. 12A and 12B shows the effects on the serum ALT and AST levels of non-alcoholic fatty liver model mice in each of administration groups and prevention groups.

FIGS. 13A and 13B shows the effects on the serum total cholesterol and low-density lipoprotein cholesterol levels of non-alcoholic fatty liver model mice in each of administration groups and prevention groups.

FIG. 14 shows the effects on the body weights of non-alcoholic steatohepatitis model mice in each of administration groups and prevention groups.

FIG. 15 shows the effects on the liver weights, liver indexes, and triglyceride contents of non-alcoholic steatohepatitis model mice in each of administration groups and prevention groups.

FIGS. 16A-16D shows the liver NAS scores, and scores for fibrosis staging, steatosis, balloon-like degeneration, and lobular inflammation of non-alcoholic steatohepatitis model mice in each of administration groups and prevention groups.

FIGS. 17A-17F shows HE staining images of the gallbladder in Example 9; wherein FIGS. 17A-17F are HE staining images of the gallbladder (200×); FIGS. 17A, 17B and 17C represent the body of the gallbladder, and FIGS. 17D, 17E, and 17F represent the base of the gallbladder.

FIG. 18 shows the results from the total cholesterol assay in Example 9.

### BEST MODE TO IMPLEMENT THE INVENTION

The present invention will be further explained in detail by examples below. The features and advantages of the present invention will become clearer and more apparent by these exemplary illustrations. However, the present invention is not limited to the following examples. The methods are conventional unless otherwise specified. The raw materials are available commercially unless otherwise specified.

The professional term “exemplary” herein means “used as an example, embodiment, or illustration”. Any examples described herein as “exemplary” need not be interpreted as superior to or better than other examples.

In addition, the technical features involved in different embodiments of the present invention described below can be combined with each other as long as they do not conflict with each other.

The content of the components involved in the present invention is detected according to a published method (referring to the methods documented in patent publication Nos. CN111077247A and CN110393738A).

I. Preparation Examples of Extracts from Morus alba L.

### Example 1 Preparation 1 of Extracts from Morus alba L

1000 kg of fresh Ramulus Mori (Morus Serrata Roxb., Yuesang 11) were taken and crushed, followed by addition of 4000 L water and reflux extraction under heating for 2 h to obtain extracted solutions, which were combined, and filtered for removal insoluble substances to obtain crude extracted solution. The crude extracted solution was concentrated by heating up to 4% of a solid mass percentage, and then kept at 50° C. as a loading solution for the cation resin column.

The column was loaded with 150 kg of model D113 macroporous weak acid phenylpropene-based cation resins, followed by sequential elution with 2 mol/L hydrochloric acid solution until the pH of the eluate to 4.5; 1 mol/L sodium hydroxide solution until the pH of the eluate to 8.5; and 2 mol/L hydrochloric acid solution until the pH of the eluate to 4.5; and a rinse with deionized water 5 times the volume of the column again to complete the activation. The concentrated extracted solution was loaded, followed by elution with 1000 L of 2.5 mol/L aqueous ammonia at an elution rate of 6 BV/h, the eluate from which was collected when the pH of the effluent from the cation column is greater than 7. When the volume of the collected solution reached 900 L, the collection was stopped, and then the collected solution was purified directly through the anion column.

The column was loaded with 62.5 kg of model D218 macroporous strong alkaline acrylic-based anion resins, followed by sequential elution with 1.5 mol/L sodium hydroxide solution until the pH of the eluate to 9.0; 1.5 mol/L hydrochloric acid solution until the pH of the eluate to 3.5; and 1.5 mol/L sodium hydroxide solution until the pH of the eluate to 9.0, to complete the activation. The collected eluate from the cation resin was loaded onto the anion resin, and the effluent was collected until its volume up to 870 L.

The collected solution was centrifuged for impurity removal, and then concentrated with a reverse ion osmosis membrane to obtain concentrated liquid with a specific gravity of 1.25, which was transferred to a tank for alcohol precipitation with addition of 25 L of anhydrous ethanol under stirring at 500 rpm. The stirring was stopped after completion of the ethanol addition. After alcohol precipitation for 24 h, the supernatant was taken and concentrated under reduced pressure to obtain an extractum.

The effluent was concentrated under reduced pressure to obtain the Ramulus Mori extractum with a percentage by mass of 52% for alkaloids (the percentage by mass of 69.5% for DNJ, 11.5% for DAB, and 15% for FAG in alkaloids), 22% for polysaccharides, 0.8% for flavonoids, and 20% for amino acids.

### Example 2 Preparation 2 of Extracts from Morus alba L

10 kg of fresh Ramulus Mori (Sangteyou 2) were taken and crushed, followed by addition of 150 L water in 2 batches and extraction by decocting for 3 h for each batch to obtain extracted solutions, which were combined, and filtered for removal of insoluble substances. The extracts were concentrated by heating up to 8% of a solid mass percentage, which was transferred to a tank for alcohol precipitation with addition of 2367.9 g of anhydrous ethanol (3 L) under stirring at 300 rpm. The stirring was stopped after completion of the ethanol addition. After alcohol precipitation for 24 h, the supernatant was taken as the loading solution for the cation resin column. The column was loaded with 5 kg of model 002SC strong acid styrene-based cation resins, which were activated according to the methods in Example 1. The concentrated extracted solution after alcohol precipitation was loaded, followed by elution with 100 L of 5 mol/L potassium chloride at an elution rate of 5 BV/h, the eluate from which was collected when white precipitates were generated in the effluent by detection with 20% silicotungstic acid. When the volume of the collected solution reached 25 L, the collection was stopped, and then the collected solution was purified directly through the anion column.

The column was loaded with 10 kg of model 711 strong alkaline styrene-based anion resins, which were activated according to the methods in Example 1. The eluate collected from the cation resin was loaded onto the anion resin, and collection of the effluent is not ended until its volume up to 15 L. The collected solution was reloaded onto cation resin and separated twice again using cation resin and anion resin in sequence according to the above methods.

The collected solution obtained after three separations on columns was centrifuged for impurity removal, and then concentrated with a reverse ion osmosis membrane to obtain concentrated solution with a specific gravity of 1.25, which was transferred to a tank for alcohol precipitation with addition of 125 g of anhydrous ethanol under stirring at 1000 rpm. The stirring was stopped after completion of the ethanol addition. After alcohol precipitation for 24 h, the supernatant was taken and concentrated under reduced pressure to obtain an extractum. Additionally, fresh Cortex Mori and Folium Mori (Santeyou 2) were extracted using the same extraction method and parameters as described above.

Extracts from Ramulus Mori were obtained with a percentage by mass of 98% for alkaloids, 0.2% for polysaccharides, 0.05% for flavonoids, and 0 for amino acids.

Extracts from Cortex Mori were obtained with a percentage by mass of 95% for alkaloids, 2% for polysaccharides, 0.1% for flavonoids, and 1% for amino acids.

Extracts from Folium Mori were obtained with a percentage by mass of 90% for alkaloids, 4% for polysaccharides, 0.1% for flavonoids, and 3% for amino acids.

### Example 3 Preparation 3 of Extracts from Morus alba L

1000 kg of fresh Ramulus Mori (Morus atropurpurea Roxb.) were taken and crushed, followed by addition of 11500 L water and reflux extraction under heating for 2 h to obtain extracted solutions, which were combined, and filtered for removal of insoluble substances to obtain crude extracted solution. The crude extracted solution was centrifuged for impurity removal, and then concentrated with a reverse ion osmosis membrane up to 1% of a solid mass percentage, which was used as a loading solution for the cation resin column.

The column was loaded with 300 kg of model D001 macroporous strong acid styrene-based cation resins, which were activated according to the methods in Preparation Example 1. The crude extracted solution after concentration was loaded, followed by elution with 5000 L of 0.04 mol/L ammonium nitrate at an elution rate of 5 BV/h, the eluate from which was collected when white precipitates were generated in the effluent by detection with 20% silicotungstic acid. When the volume of the collected solution reached 1000 L, the collection was stopped.

The collected solution obtained after cation column separation was concentrated with a nanofiltration membrane, followed by concentration under reduced pressure to obtain an extractum.

Extracts from Ramulus Mori were obtained with a percentage by mass of 15% for alkaloids, 20% for polysaccharides, 7% for flavonoids, and 45% for amino acids.

### Example 4 Preparation 4 of Extracts from Morus alba L

333 kg of dry Ramulus Mori (Yuesang 11) were taken and crushed, followed by addition of 4000 L water and reflux extraction under heating in two batches, with each reflux for 1 h to obtain extracted solutions, which were combined, filtered and concentrated to 1 kg of raw medicine/L.

The column was loaded with 150 kg of model D113 macroporous weak acid phenylpropene-based cation resins, followed by sequential elution with 2 mol/L hydrochloric acid solution until the pH of the eluate to 4.5, 1 mol/L sodium hydroxide solution until the pH of the eluate to 8.5, and 2 mol/L hydrochloric acid solution until the pH of the eluate to 4.5, and then a rinse with deionized water 5 times the volume of the column again to complete the activation. The concentrated extracted solution was loaded, followed by elution with 1000 L of 2.5 mol/L aqueous ammonia at a elution rate of 6 BV/h, the eluate from which was collected when the pH of the effluent from the cation column was greater than 7. When the volume of the collected solution reached 900 L, the collection was stopped, and then the collected solution was purified directly through the anion column.

The column was loaded with 125 kg of model D218 macroporous strong alkaline acrylic based anion resins, followed by sequential elution with 1.5 mol/L sodium hydroxide solution until the pH of the eluate to 9.0; 1.5 mol/L hydrochloric acid solution until the pH of the eluate to 3.5; and 1.5 mol/L sodium hydroxide solution until the pH of the eluate to 9.0, to complete the activation. The collected eluate from the cation resin was loaded onto the anion resin with collection of the effluent with a pH greater than 8 until its volume up to 870 L.

The collected solution obtained after separation on the anion column was centrifuged for impurity removal, and then filtered with a microfiltration membrane for impurity removal and concentrated with a reverse ion osmosis membrane to obtain concentrated liquid with a specific gravity of 1.1, which was transferred to a tank for alcohol precipitation with addition of 15 kg of anhydrous ethanol under stirring at 400 rpm. The stirring was stopped after completion of the ethanol addition. After alcohol precipitation for 24 h, the supernatant was taken and concentrated under reduced pressure to obtain an extractum from Ramulus Mori. The sample content was as follows: a percentage by mass of 80% for alkaloids, 5% for polysaccharides, 0.1% for flavonoids, and 4% for amino acids.

### Example 5 Preparation 5 of Extracts from Morus alba L

400 kg of dry Ramulus Mori (Yuesang 11) were taken and crushed, followed by addition of 4000 L water and reflux extraction under heating in two batches, with each reflux for 1 h to obtain extracted solutions, which were combined, filtered and concentrated to 1 kg of raw medicine/L.

The column was loaded with 62.5 kg of model D218 macroporous strong alkaline acrylic based anion resins, followed by sequential elution with 1.5 mol/L sodium hydroxide solution until the pH of the eluate to 9.0; 1.5 mol/L hydrochloric acid solution until the pH of the eluate to 3.5; and 1.5 mol/L sodium hydroxide solution until the pH of the eluate to 9.0, to complete the activation. The collected extracted concentrate was loaded onto the anion resin and the effluent was collected.

The collected solution obtained after anion column separation was filtered with a microfiltration membrane for impurity removal and concentrated with a reverse ion osmosis membrane, followed by concentration under reduced pressure and drying to obtain the extractum from Ramulus Mori. The sample content was as follows: a percentage by mass of 3% for alkaloids, 70% for polysaccharides, 10% for flavonoids, and 10% for amino acids.

### Example 6 Preparation 6 of Extracts from Morus alba L

1500 kg of fresh Ramulus Mori (Morus Serrata Roxb., Yuesang 11) were taken and crushed, followed by addition of 6000 L water and reflux extraction under heating for 2 h to obtain extracted solutions, which were combined, and filtered with removal of insoluble substances to obtain crude extracts. The crude extracts were concentrated by heating up to 4% of a solid mass percentage, and then kept at 50° C. as the loading solution for the cation resin column.

The column was loaded with 100 kg of model D113 macroporous weak acid phenylpropene-based cation resins, followed by sequential elution with 2 mol/L hydrochloric acid solution until the pH of the eluate to 4.5, 1 mol/L sodium hydroxide solution until the pH of the eluate to 8.5, and 2 mol/L hydrochloric acid solution until the pH of the eluate to 4.5, and then a rinse with deionized water 5 times the volume of the column again to complete the activation. The concentrated extracted solution was loaded, followed by elution with 1000 L of 2.5 mol/L aqueous ammonia at an elution rate of 6 BV/h, the eluate from which was collected when the pH of the effluent from the cation column greater than 7. When the volume of the collected solution reached 900 L, the collection was stopped, and then the collected solution was purified directly through the anion column.

The column was loaded with 62.5 kg of model D218 macroporous strong alkaline acrylic based anion resins, followed by sequential elution with 1.5 mol/L sodium hydroxide solution until the pH of the eluate to 9.0; 1.5 mol/L hydrochloric acid solution until the pH of the eluate to 3.5; and 1.5 mol/L sodium hydroxide solution until the pH of the eluate to 9.0, to complete the activation. The collected eluate from the cation resin was loaded onto an anion resin with collection of the effluent until its volume up to 870 L. The extractum from Ramulus Mori were obtained by concentrating the effluent under reduced pressure with a percentage by mass of 30% for alkaloids, 35% for polysaccharides, 2% for flavonoids, and 25% for amino acids.

### Example 7 Preparation 7 of Extracts from Morus alba L

1000 kg of fresh Ramulus Mori (Morus Serrata Roxb., Yuesang 11) were taken and crushed, followed by addition of 4000 L water and reflux extraction under heating for 2 h to obtain extracted solutions, which were combined, and filtered for removal of insoluble substances to obtain crude extracts. The crude extracted solution was concentrated by heating up to 4% of a solid mass percentage, and then kept at 50° C. as the loading solution for the cation resin column.

The column was loaded with 100 kg of model D113 macroporous weak acid phenylpropene-based cation resins, followed by sequential elution with 2 mol/L hydrochloric acid solution until the pH of the eluate to 4.5; 1 mol/L sodium hydroxide solution until the pH of the eluate to 8.5; and 2 mol/L hydrochloric acid solution until the pH of the eluate to 4.5; and then a rinse with deionized water 5 times the volume of the column again to complete the activation. The extracted solution after concentration was loaded, followed by elution with 1000 L of 2.5 mol/L aqueous ammonia at a elution rate of 6 BV/h, the eluate from which was collected when the detected pH of the effluent from the cation column was greater than 7. When the volume of the collected solution reached 900 L, the collection was stopped, and then the collected solution was purified directly through the anion column.

The column was loaded with 62.5 kg of model D218 macroporous strong alkaline acrylic based anion resins, followed by sequential elution with 1.5 mol/L sodium hydroxide solution until the pH of the eluate to 9.0; 1.5 mol/L hydrochloric acid solution until the pH of the eluate to 3.5; and 1.5 mol/L sodium hydroxide solution until the pH of the eluate to 9.0, to complete the activation. The collected eluate from the cation resin was loaded onto the anion resin with collection of the effluent until its volume up to 870 L. Extractum from Ramulus Mori were obtained by concentrating the effluent under reduced pressure with a percentage by mass of 40% for alkaloids, 25% for polysaccharides, 0.5% for flavonoids, and 25% for amino acids.

### Example 8 Preparation 8 of Extracts from Morus alba L

333 kg of dry Ramulus Mori (Yuesang 11) were taken and crushed, followed by addition of 4000 L water and reflux extraction under heating in two batches, with each reflux for 1 h to obtain extracted solutions, which were combined, filtered and concentrated to 1 kg of raw medicine/L.

The column was loaded with 150 kg of model D113 macroporous weak acid phenylpropene-based cation resins, followed by sequential elution with 2 mol/L hydrochloric acid solution until the pH of the eluate to 4.5; 1 mol/L sodium hydroxide solution until the pH of the eluate to 8.5; and 2 mol/L hydrochloric acid solution until the pH of the eluate to 4.5; and then a rinse with deionized water 5 times the volume of the column again, to complete the activation. The extracted solution after concentration was loaded, followed by elution with 1000 L of 2.5 mol/L aqueous ammonia at a elution rate of 6 BV/h, the eluate from which was collected when the detected pH of the effluent from the cation column was greater than 7. When the volume of the collected solution reached 900 L, the collection was stopped, and then the collected solution was purified directly through the anion column.

The column was loaded with 62.5 kg of model D218 macroporous strong alkaline acrylic based anion resins, followed by sequential elution with 1.5 mol/L sodium hydroxide solution until the pH of the eluate to 9.0; 1.5 mol/L hydrochloric acid solution until the pH of the eluate to 3.5; and 1.5 mol/L sodium hydroxide solution until the pH of the eluate to 9.0, to complete the activation. The collected eluate from the cation resin was loaded onto the anion resin with collection of the effluent with a pH greater than 8 until its volume up to 870 L.

The collected solution obtained after separation on the anion column was filtered with a microfiltration membrane for impurity removal and concentrated with a reverse ion osmosis membrane to obtain concentrated solution with a specific gravity of 1.1, which was transferred into a tank for alcohol precipitation with addition of 15 kg of anhydrous ethanol under stirring at 400 rpm. The stirring was stopped after completion of the ethanol addition. After alcohol precipitation for 24 h, the supernatant was taken and concentrated under reduced pressure to obtain the extractum from Ramulus Mori. The sample content was as follows: a percentage by mass of 63% for alkaloids, 23% for polysaccharides, 1% for flavonoids, and 5% for amino acids.

II. Validation of the Effect of Extracts from Morus alba L.

### Experimental Example 1: Pharmacodynamic Experiments of Extracts from Morus alba L

### 1.1 Cellular Level

### 1.1.1 HepG2 Hepatocytes

HepG2 hepatocytes were cultured overnight in a 6-well plate at a seeding density of 2×10⁵ cells/ml after digestion. HepG2 cells were treated with palmitic acid (PA) to create a non-alcoholic fatty liver model, with 0.25% BSA treatment as a control. Cells in the model groups were separately administrated with extracts from Morus alba L. (designed to be 25 μg/ml (containing 17 μg/ml DNJ) based on total alkaloids (SZ-A)) obtained from Example 1, 20 μg/ml DNJ, 5 μg/ml FAG, or 5 μg/ml DAB, and cultured for 24 h. Cells were lysed with the lysis solution containing Triton x-100 after removal of the supernatant, and the triglyceride (TG) and cholesterol (TC) in hepatocytes were detected using a kit.

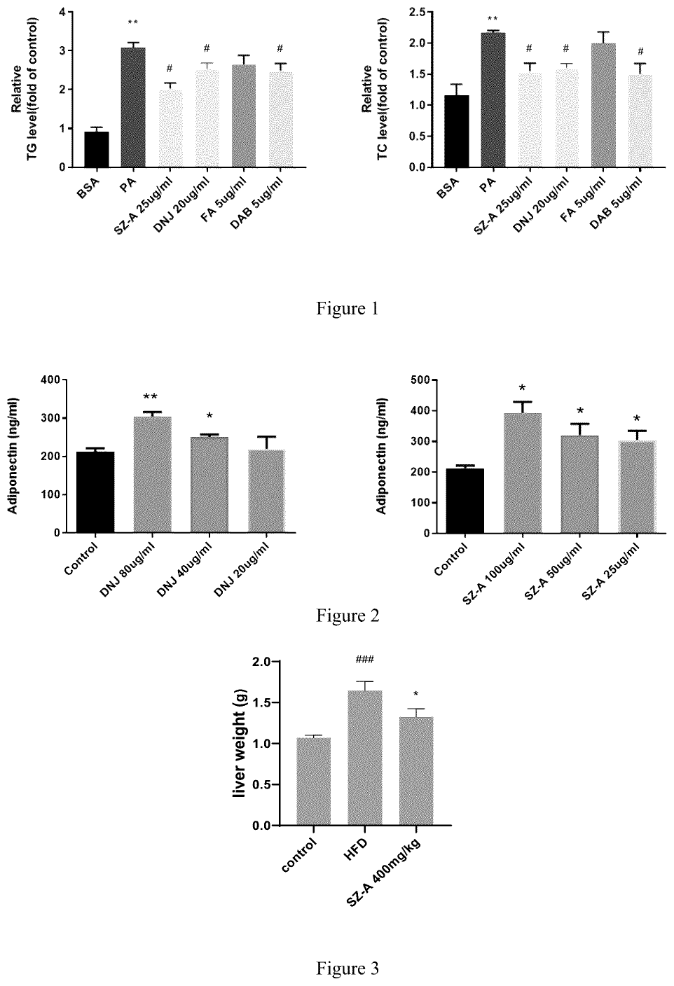

The results were shown in FIG. 1, which showed that palmitic acid stimulation of cells might cause an increase in liver triglyceride and cholesterol levels. Both SZ-A 25 μg/ml and DNJ 20 μg/ml might reduce the triglyceride level, and SZ-A had a better effect, that is to say, other ingredients than DNJ in SZ-A might also play a role. SZ-A 25 μg/ml reduced the cholesterol level comparable to DNJ 20 μg/ml.

### 1.1.2 Effects on the Secretion of Adiponectin in 3T3-L1 Adipocytes

Reduced adiponectin levels were independent risk factors for NAFLD and liver dysfunction. Adiponectin might activate liver AMPK, inhibit lipid synthesis, and promote lipid oxidation. Adiponectin might also affect inflammation of hepatic macrophages and fibrosis of hepatic stellate cells. 3T3-L1 pre-adipocytes were cultured in the medium containing calf serum and seeded into a 6-well plate at a density of 2×10⁴ cells/ml. After 48 h, the medium was refreshed, followed by replacements with the medium containing FBS, 1 uM dexamethasone, 0.5 mM 3-isobutyl-1-methylxanthine, and 10 μg/ml insulin once on days 2 and 4, respectively, and a replacement with the medium containing FBS and insulin on day 6, with addition of different concentrations of extracts from Morus alba L. obtained from Example 1 (100 μg/ml, 50 μg/ml, and 25 μg/ml based on total alkaloid SZ-A) or DNJ (80 ug/ml, 40 μg/ml, and 20 μg/ml). The supernatant was collected after culture for 8 days, and the concentration of adiponectin in the supernatant was detected using an ELISA kit. The results were shown in FIG. 2. It might be seen from FIG. 2 that 100 μg/ml SZ-A (containing 69.5 ug/ml DNJ) could more effectively increase the concentration of adiponectin compared to 80 μg/ml DNJ, and the same for other concentrations of SZ-A.

### 1.2 Animal Experiments

Mice were fed with the high-fat diet to construct an NFALD model and administered with extracts from Morus alba L. prepared in Preparation Example 1.

Forty five healthy 6-week-old male C57 mice were randomly assigned to the normal group (Chow), model group (HFD), and SZ-A group, with 15 mice in each group. Mice in the normal group were fed with basic diet, and mice in the model group and SZ-A group were fed with high-fat diet (Research Diet, D12492, 60 kcal % Fat) to establish a non-alcoholic fatty liver model in mice. After being fed for 14 weeks, mice in each group were gavaged with corresponding drugs every day for 6 consecutive weeks administration. Mice in the SZ-A group were gavaged at a dose of 400 mg/kg/d based on total alkaloids in the extracts, and the model group maintained high-fat feeding while being administrated. Mice in the normal and model group were gavaged with solvents at corresponding doses. During drug treatment, the general conditions of the mice were observed. All mice were fasted for 12 h after the last administration, followed by weighting and collecting blood from the eyeballs, from which serum was separated by centrifugation. The levels of low-density lipoprotein (LDL), high-density lipoprotein (HDL), aspartate aminotransferase (AST), alanine aminotransferase (ALT), and total cholesterol (TC) in serum were detected using a fully automated biochemical analyzer. Mice were then euthanized with livers taken for calculation of the liver index (liver weight/body weight) and observation of the pathological changes of liver tissues using HE staining and Oil Red O staining methods. Part of the livers was homogenized to detect the levels of triglyceride (TG) and total cholesterol (TC) in liver tissues.

### Experimental Results:

### (1) Liver Weight and Liver Index

### SZ-A Might Reduce Liver Weight and Liver Index

The liver weights of mice in each group after administration were shown in FIG. 3. As shown in FIG. 3, the liver weight of mice in the SZ-A administration group was significantly lower than that in the model group.

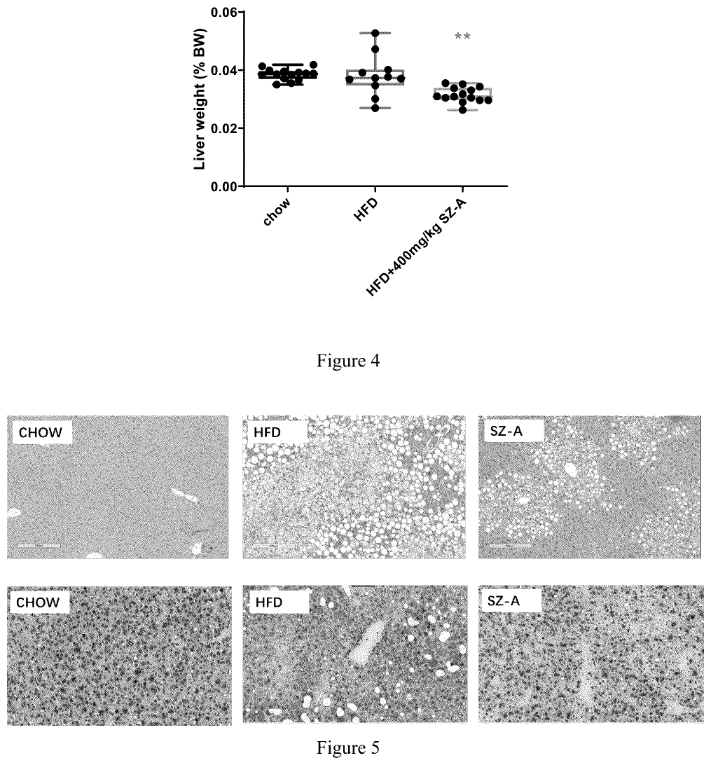

The liver indexes of mice in each group after administration were shown in FIG. 4. As shown in FIG. 4, the liver index of mice in the SZ-A administration group was significantly lower than that in the model group.

### (2) Detection of Liver Lipids

H&E staining: fresh liver tissues from each group were fixed in 4% paraformaldehyde solution for 48 h, and then placed in different concentrations of alcohol for gradient dehydration, followed by permeabilization in xylene. The permeabilized tissues were embedded in paraffin wax. The embedded wax block was fixed on the slicer for slicing. The nucleus and intracellular ribosomes may be stained blue with hematoxylin, and the cytoplasm might be stained red with eosin.

Detection of triglyceride (TG) and cholesterol (TC) in liver tissues: fresh liver tissues were homogenized and lysed in the tissue homogenate, followed by detection using a triglyceride and cholesterol assay kit.

The results from H&E and Oil Red O staining were shown in FIG. 5. As shown in FIG. 5, SZ-A may reduce lipid accumulation in the liver.

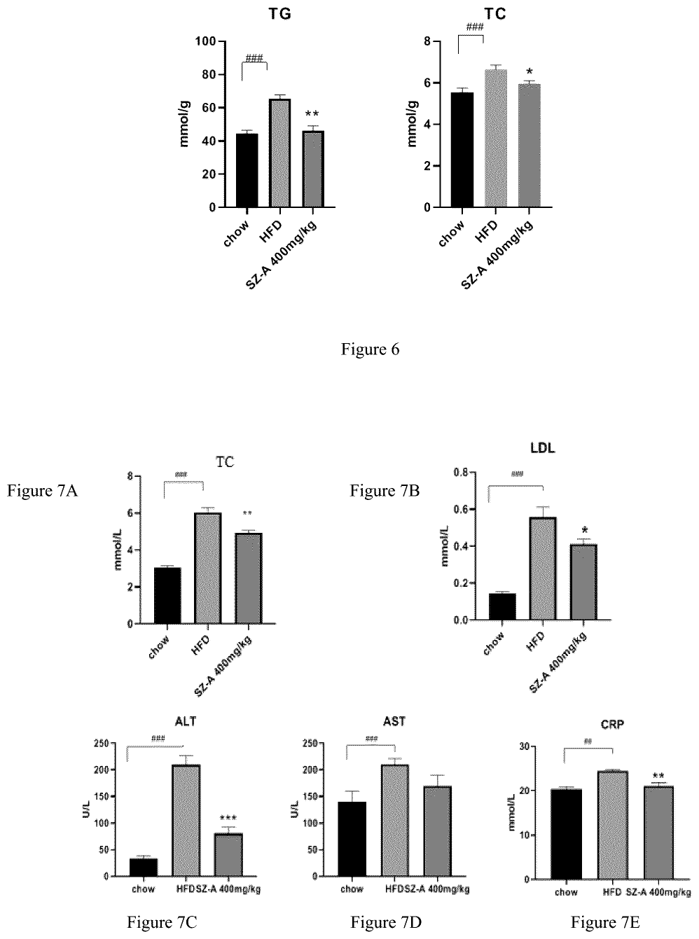

The results from triglyceride and cholesterol detection were shown in FIG. 6. As shown in FIG. 6, SZ-A may reduce the levels of triglyceride and cholesterol in the liver.

The results showed that SZ-A may significantly reduce the levels of triglyceride and cholesterol in the liver, and alleviate lipid deposition in the liver.

### (3) Blood Biochemical Test:

Serum was took from mice and used to detect ALT and AST activities, as well as CRP (C-reactive protein), total cholesterol (CHO), low-density lipoprotein (LDL), and high-density lipoprotein (HDL) levels by a blood biochemistry analyzer.

The results were shown in FIGS. 7A-7E. As shown in FIGS. 7A-7E, SZ-A might alleviate the high-fat diet-induced ALT increasement, reduce serum total cholesterol and LDL levels, and reduce serum CRP, with no toxicity in the liver and kidney.

### (4) Transcriptomics and Fluorescence Quantitative PCR

The liver tissues from mice were added with Trizol and homogenized for lysis, followed by addition of chloroform under shaking, and subsequent centrifugation after placement, with the upper colorless aqueous phase transferred to another tube and addition of isopropanol to mix well, and then centrifuged after placement, with the supernatant discarded, followed by addition of 75% (v/v) ethanol, which was mixed well and centrifuged with the supernatant discarded, and the pellets dried. The pellets were dissolved in DEPC treated water which was added based on the amount of RNA obtained, resulting in RNA samples. The RNA concentration was detected using Agilent 2100 Bioanalyzer, and sequenced using BGI-SEQ 500 platform if the results passed. The extracted RNA was reverse transcribed and detected by fluorescence quantitative PCR.

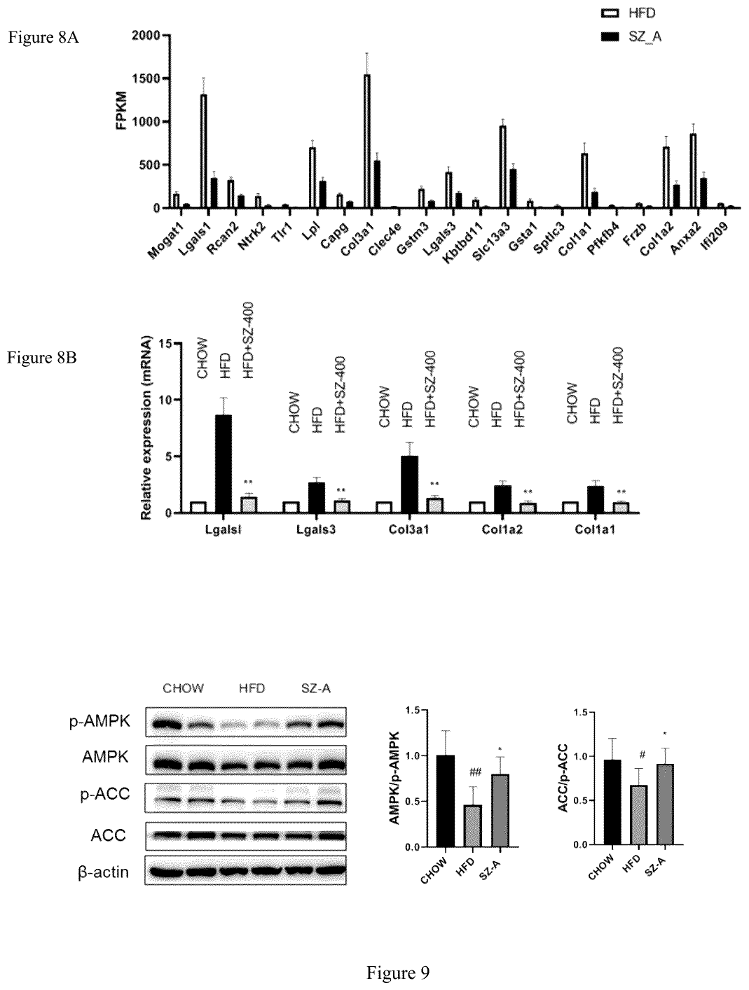

The results from transcriptomics (see FIG. 8A) showed that hepatic fibrosis-related genes such as Lgals1, Lgals3, Col3a1, Col1a1, and Col1a2 were mainly changed, compared with the control group.

The above results were further validated by fluorescence quantitative PCR (see FIG. 8B). As shown in the figure, SZ-A might significantly decrease the expression of the hepatic fibrosis-related genes such as Lgals1, Lgals3, Col3a1, Col1a1, and Col1a2.

### (5) Changes in Liver Signaling Pathways

Fresh liver tissues were homogenized to extract proteins for detection of proteins such as p-AMPK, AMPK, p-ACCC, and ACC using western blot.

The results were shown in FIG. 9. As shown in FIG. 9, compared with the HFD group, SZ-A might increase the expression levels of p-AMPK and p-ACC, thereby inhibiting fatty acid synthesis and promoting fatty acid oxidation.

### 1.3 Administration at Different Doses

Treatment with extracts from Morus alba L. prepared in Example 1 at different doses:

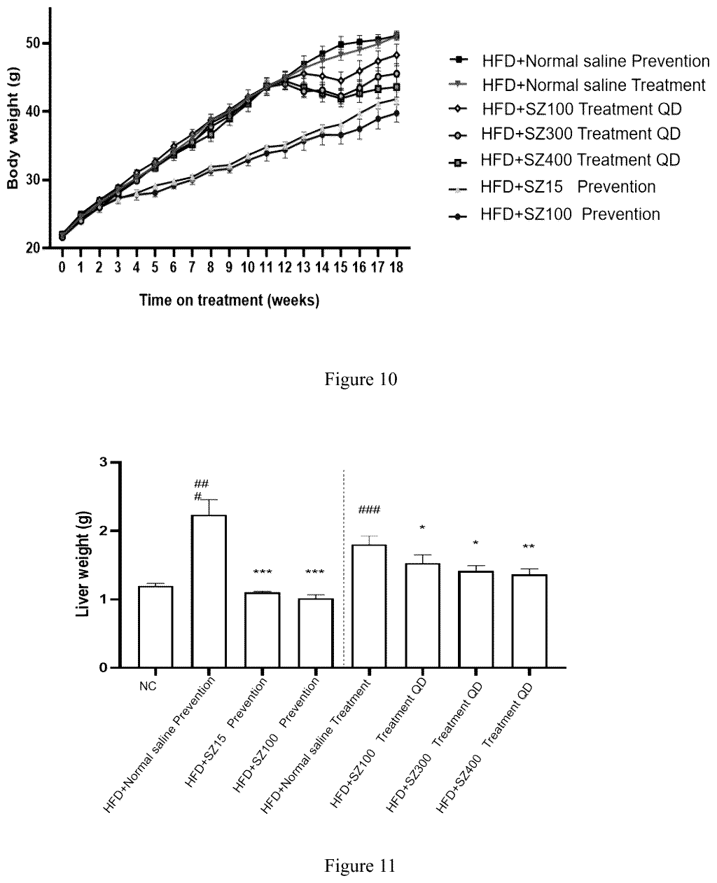

Healthy 6-week-old male C57 mice were randomly assigned to the normal group (NC), model group (HFD), and SZ-A-100, SZ-A-300 and SZ-A-400 groups. Mice in the normal group were fed with basic diet, and mice in the model group and SZ-A groups were fed with high-fat diet (Research Diet, D12492, 60 kcal % Fat) to establish a non-alcoholic fatty liver model in mice. After being fed for 12 weeks, mice were orally administered with the extracts from Example 1 for 6 consecutive weeks, while continuing to feed with the corresponding diet. The specific animal grouping and drug treatment were shown in the table below. The sampling and detection methods were shown in section 1.2 in experimental Example 1, and the results were shown in FIGS. 10-13B.

Prevention with extracts from Morus alba L. prepared in Example 1 at different doses:

Healthy 6-week-old male C57 mice were randomly assigned to the normal group (NC), model group (HFD), and SZ-A-15 and SZ-A-100 groups. Mice in the normal group were fed with basic diet, and mice in the model group and SZ-A groups were fed with high-fat diet (Research Diet, D12492, 60 kcal % Fat) to establish a non-alcoholic fatty liver model in mice. Mice, starting from 6-week old, were orally administered with the extracts from Example 1 at the doses shown in Table 2 for 18 weeks, while continuing to feed with the corresponding diet. The sampling and detection methods were shown in section 1.2 in experimental Example 1, and the results were shown in FIGS. 10-13B.

TABLE 1

| | | | Administration | | | | |
|---|---|---|---|---|---|---|---|
| | | | Dose | | | | |
| | | | (mg/kg), | | | | |
| | | | based on total | Number of | Mode of | Frequency of | |
| | Group | Treatment | alkaloid | animals | administration | administration | |
| Normal | 1 | Normal | | 15 | Oral | | |
| diet | | feed (NC) | | | | | |
| HFD | 2 | Normal | | 10 | Oral | Twice a day | Treatment |
| | | saline | | | | (Interval | |
| | | (HFD) | | | | of 8 h) | |
| | 3 | SZ-A base | 100 | 10 | Oral | Single | Treatment |
| | 4 | SZ-A base | 300 | 10 | Oral | Single | Treatment |
| | 5 | SZ-A base | 400 | 10 | Oral | Single | Treatment |
| | 6 | SZ-A base | 15 | 5 | Oral | Single | Prevention |
| | 7 | SZ-A base | 100 | 5 | Oral | Single | Prevention |
| | 8 | Normal | | 5 | Oral | Single | Prevention |
| | | saline | | | | | |

As could be seen from FIG. 10, in terms of weight reduction, the body weights of mice in the SZ-A100, SZ-A 300, and SZ-A 400 treatment groups were significantly lower than that in the model group after administration. The body weights of mice in the SZ-A15 and SZ-A100 prevention groups were significantly lower than that in the model group after administration.

From FIG. 11, it could be seen that in terms of liver weight reduction, the liver weights of mice in the SZ-A100, SZ-A 300, and SZ-A 400 treatment groups all were significantly lower than that in the model group. The liver weights of mice in the SZ-A15 and SZ-A100 prevention groups were significantly lower than that in the model group, with the best preventive effect in the SZ-A15 group.

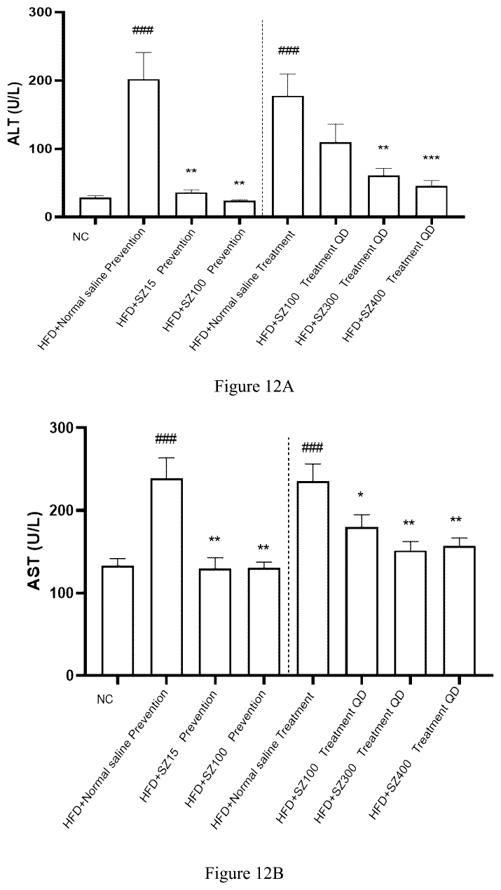

From FIGS. 12A and 12B, it could be seen that the ALT levels of mice in the SZ-A100, SZ-A 300, and SZ-A 400 treatment groups were significantly lower than that in the model group. The ALT levels of mice in the SZ-A15 and SZ-A100 prevention groups were significantly lower than that in the model group. The AST levels of mice in the SZ-A100, SZ-A 300, and SZ-A 400 treatment groups were significantly lower than that in the model group. The AST levels of mice in the SZ-A15 and SZ-A100 prevention groups were significantly lower than that in the model group.

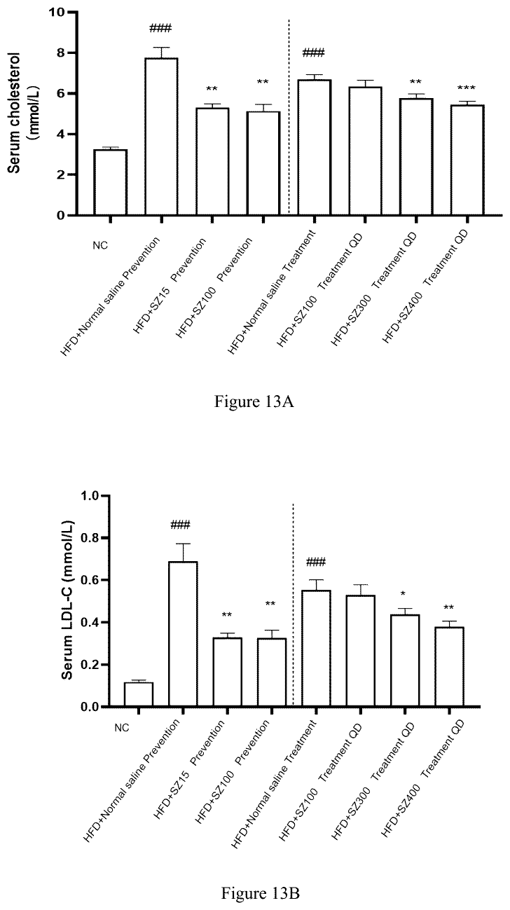

From the serum total cholesterol results in FIGS. 13A and 13B, it could be seen that the serum total cholesterol levels of mice in the SZ-A 300 and SZ-A 400 treatment groups were significantly lower than that in the model group; and there was no significant difference in serum total cholesterol levels between the SZ-A100 treatment group and the model group. The serum total cholesterol levels of mice in the SZ-A15 and SZ-A 100 prevention groups were significantly lower than that in the model group.

From the serum low-density lipoprotein cholesterol results in FIGS. 13A-13B, it could be seen that the serum low-density lipoprotein cholesterol levels of mice in the SZ-A 300 and SZ-A 400 treatment groups were significantly lower than that in the model group; and there was no significant difference in serum low-density lipoprotein cholesterol levels between the SZ-A100 treatment group and the model group. The serum total cholesterol levels of mice in the SZ-A15 and SZ-A100 prevention groups were significantly lower than that in the model group.

1.4 Effect of Extracts from Morus alba L. On GAN Diet-Induced Non-Alcoholic Steatohepatitis (NASH) Animal Model in C57BL/6 Mice

Healthy 6-week-old male C57BL/6J mice were selected and assigned to the model group, low-dose group SZ-A100 (100 mg/kg/d, based on alkaloids), medium dose group SZ-A200 (200 mg/kg/d, based on alkaloids), and high-dose group SZ-A300 (300 mg/kg/d, based on alkaloids). At the same time, the normal feed group (normal saline) was set up. The model group and SZ-A group mice were fed with GAN diet (Research Diet, D09100310) to establish a non-alcoholic steatohepatitis (NASH) animal model. After 20 weeks of induction, mice were continuously administered (all are extracts from Example 1) for six weeks, and the corresponding feed was continued during the treatment period. The specific animal grouping and drug treatment were shown in the table below.

TABLE 2
Animal grouping and drug treatment

| | | | Frequency of | Adminis- |
|---|---|---|---|---|
| | | Number of | adminis- | tration |
| Grouping | Group | animals | tration | route |
| 1 | Normal feed group | 8 | Twice a day | Oral |
| 2 | Model group | 8 | Twice a day | Oral |
| 3 | SZ-A100 dose group | 8 | Twice a day | Oral |
| 4 | SZ-A200 dose group | 8 | Twice a day | Oral |
| 5 | SZ-A300 dose group | 8 | Twice a day | Oral |

Note:
mice were administered twice a day with 8-hour interval.

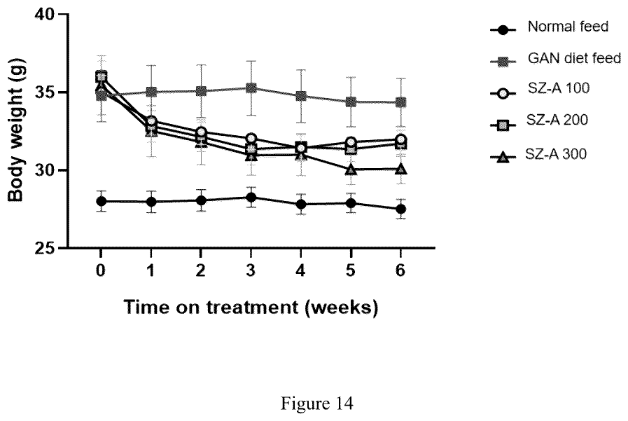

From FIG. 14, it could be seen that compared with the model group, the body weight of the mice in the treatment group fluctuated first and then steadily decreased, with the best weight loss effect in the SZ-A300 group.

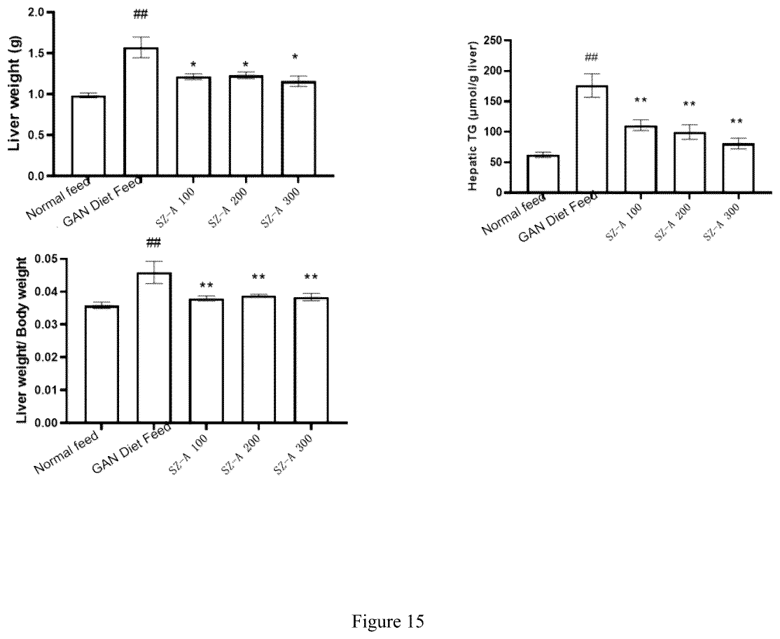

From FIG. 15, it could be seen that compared with the model group, SZ-A100, SZ-A200, and SZ-A300 all had significantly reduced liver weights and triglyceride levels in the livers.

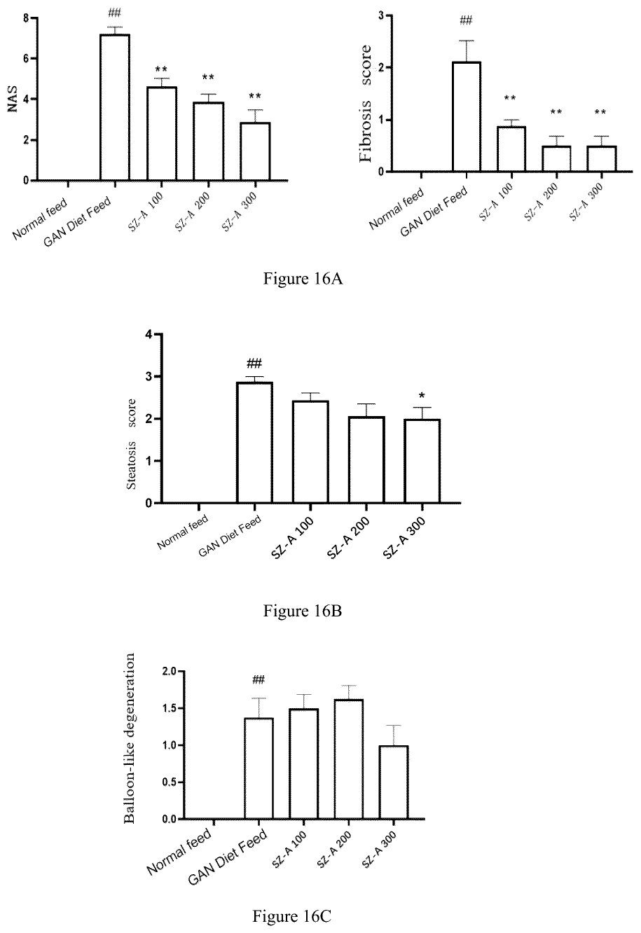

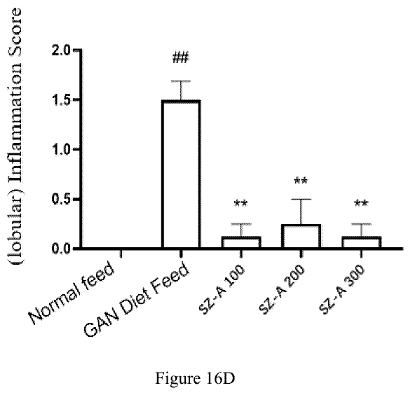

From FIGS. 16A-16D, it could be seen that compared with the model group, SZ-A100, SZ-A200, and SZ-A300 all could significantly reduce NAS scores, with the best effect for SZ-A300. Compared with the model group, SZ-A100, SZ-A200, and SZ-A300 could significantly reduce the scores for fibrosis, with the best effect for SZ-A200 and SZ-A300. At the same time, it could also be seen from FIGS. 16A-16D that SZ-A300 significantly reduced the score for steatosis; SZ-A300 could improve balloon-like degeneration; SZ-A100, SZ-A200, and SZ-A300 all could significantly alleviate the scores for lobular inflammation, and there was no statistically significant difference among the three groups. That was to say, SZ-A300 had an improving effect on steatosis, balloon-like degeneration, and lobular inflammation.

The NAS scoring criteria in FIGS. 16A-16D were shown in Table 3 below.

TABLE 3
Liver pathology scoring criteria
NAS Scoring (AASLD Guidelines)

| Lesions | Scores | Descriptions |
|---|---|---|
| Steatosis | 0 | <5% |
| | 1 | 5%~33% |
| | 2 | 34%~66% |
| | 3 | >66% |
| (lobular) | 0 | 0, (counting the number of necrotic |
| Inflammation | | lesions at 20 magnifications) |
| | 1 | <2, (counting the number of necrotic |
| | | lesions at 20 magnifications) |
| | 2 | 2~4, (counting the number of |
| | | necrotic lesions at 20 magnifications |
| | 3 | >4, (counting the number of necrotic |
| | | lesions at 20 magnifications) |
| Balloon-like | 0 | None |
| degeneration | 1 | Few balloon-like cells |
| | 2 | Numerous balloon-like cells |
| Fibrosis | 0 | None |
| staging | 1a | Mild perisinusoidal fibrosis in acinar |
| | | zone 3 |
| | 1b | Moderate perisinusoidal fibrosis in |
| | | acinar zone 3 |
| | 1c | Only periportal vein fibrosis |
| | 2 | 1a/1b+ Only periportal vein fibrosis |
| | 3 | Bridging fibrosis |
| | 4 | Hepatic cirrhosis |

### Experimental Examples 2-8

The drugs in section 1.2 or 1.3 of experimental Example 1 were replaced with extracts from Morus alba L in Preparation 2-8, where extracts from Ramulus Mori in Preparation 2, with other steps remained unchanged. The detection results of triglyceride (TG) and cholesterol (TC) in the liver tissues showed that the extracts from Morus alba L. in Preparations 2-8 all could significantly reduce triglyceride (TG) and cholesterol (TC) levels in the liver tissues.

### Experimental Example 9

### 1. Construction of Animal Models

Mice were fed with the high-fat diet to construct a model and administered with extracts from Morus alba L. prepared in Preparation 1.

Forty five healthy 6-week-old male C57 mice were randomly assigned to the normal group (Chow), model group (HFD), and SZ-A group, with 15 mice in each group. Mice in the normal group were fed with basic diet, and mice in the model group and SZ-A group were fed with high-fat diet (Research Diet, D12492, 60 kcal % Fat) to establish a model in mice. After being fed for 14 weeks, mice in each group were administered with corresponding drugs every day for 6 consecutive weeks of administration. Mice in the SZ-A group were injected intraperitoneally at a dose of 200 mg/kg/d based on total alkaloids, and the mice in the model group were maintained high-fat feeding while being administrated. Mice in the normal and model group were injected with solvents (normal saline) at corresponding doses. During drug treatment, the general conditions of the mice were observed.

2. Serum separation: the whole blood of mice was collected into a tube by removing the eyeballs, and then the blood was centrifuged at 3000 rpm for 10 min after placement, with serum taken and transferred to another new tube. They were stored at −80° C. for further analysis.

3, HE staining for gallbladder: fresh gallbladder tissues from each group were fixed in 4% paraformaldehyde solution for 48 h, and then placed in different concentrations of alcohols for gradient dehydration, followed by permeabilization in xylene. The permeabilized tissues were embedded in paraffin wax. The embedded wax block was fixed on the slicer for slicing.

4. Serum total cholesterol level assay: the cholesterol content was determined using a total cholesterol assay kit. Samples with bubbles removal were shaken well and incubated at 37° C. for 10 min, with blank wells, calibration wells, and loading wells separately set for them. The absorbance value at a wavelength of 510 nm was measured using a microplate reader. The total cholesterol concentration for each sample was calculated according to the formula.

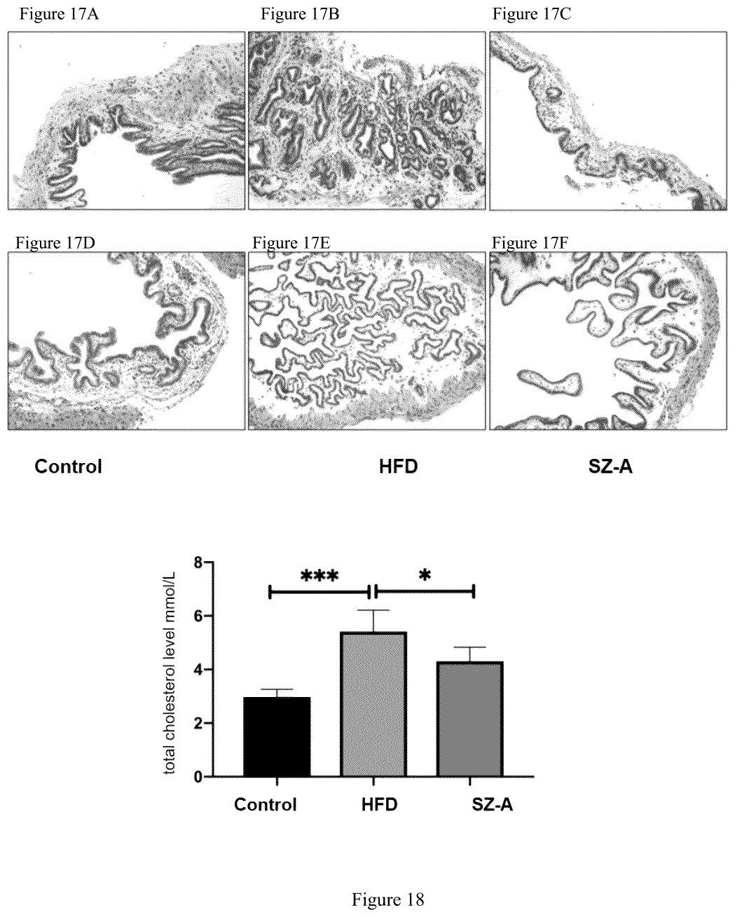

The experimental results were shown in FIGS. 17A-17F.

In the gallbladder body, compared with the control group, significantly thickened gallbladder wall, the formation of numerous Aschoff sinuses in the gallbladder epithelium, and inflammatory cells infiltration were observed in the model group. At the base of the gallbladder, the gallbladder muscle layer in the model group was disordered, with significant mucosa hyperplasia and edema and detachment. The above pathological changes were all improved in the SZ-A group.

As shown in FIG. 18, compared with the control group, the total cholesterol level was significantly increased in the model group, while significantly decreased in the SZ-A group.

The present invention was explained as above in combination with preferred embodiments, but these embodiments are only exemplary and serve only as illustrative. On this basis, various replacements and improvements can be made to the present invention, all of which fall within the scope of protection of the present invention.

### INDUSTRIAL APPLICATIONS

The extracts from Morus alba L. used in the present invention exerts multi-target pharmacological effects through multi-components, and specifically can regulate the liver lipid content by regulating the synthesis and oxidation of fatty acids, and can also affect hepatic fibrosis, which is more conducive to the treatment of non-alcoholic fatty liver disease. The extracts from Morus alba L. or main active ingredients thereof can be used to prepare a medicament for preventing and/or treating the hepatobiliary disease.

1-13. (canceled)

14. A method of alleviating, preventing and/or treating hepatobiliary disease in a subject, comprises administrating extract from Morus alba L. or the main active ingredient thereof to the subject.

15. The method according to claim 14, wherein the hepatobiliary disease is non-alcoholic fatty liver disease and/or cholecystitis.

16. The method according to claim 15, wherein the alleviating, preventing and/or treating hepatobiliary disease in the subject comprises at least one of the followings:
1) reduction of the hepatic lipid content;
2) alleviation or inhibition of hepatic fibrosis;
3) alleviation of increased ALT caused by non-alcoholic fatty liver disease;
4) inhibition of increase of total cholesterol, triglyceride, and/or LDL in the serum or liver caused by non-alcoholic fatty liver disease or non-alcoholic steatohepatitis;
5) inhibition of increase of CRP in the serum caused by non-alcoholic fatty liver disease;
6) inhibition of the expression of genes associated with hepatic fibrosis;
7) promotion of adiponectin secretion in adipocytes;
8) inhibition of fatty acid synthesis, promotion of fatty acid oxidation;
9) inhibition of increase of liver index caused by non-alcoholic fatty liver disease or non-alcoholic steatohepatitis;
10) improvement of liver NAS score for non-alcoholic steatohepatitis;
11) improvement of hepatic steatosis, balloon-like degeneration, and/or lobular inflammation caused by nonalcoholic steatohepatitis.

17. The method according to claim 15, wherein,
when the non-alcoholic fatty liver disease is treated, the extract from Morus alba L. or the main active ingredients thereof is administered at a single dose of 100-400 mg/kg body weight for a mouse and at a single dose of 500-2000 mg/kg body weight for human, based on total alkaloids.

18. The method according to claim 15, wherein,
when the non-alcoholic fatty liver disease is prevented, the extract from Morus alba L. or the main active ingredients thereof is administered at a single dose of 15-100 mg/kg body weight for a mouse, and at a single dose of 75-500 mg/kg body weight for human, based on total alkaloids.

19. The method according to claim 14, wherein,
When the non-alcoholic steatohepatitis is treated, the extract from Morus alba L. or the main active ingredients thereof is administered at a single dose of 100-400 mg/kg body weight for a mouse, and at a single dose of 500-2000 mg/kg body weight for human, based on total alkaloids.

20. A method for reducing the hepatic lipid content, alleviating or inhibiting hepatic fibrosis, alleviating increased ALT caused by non-alcoholic fatty liver; reducing total cholesterol, triglyceride and/or LDL in the serum or liver; reducing CRP in the serum; improving liver NAS score; or improving hepatic steatosis, balloon-like degeneration and/or lobular inflammation in a subject with non-alcoholic fatty liver disease and/or non-alcoholic steatohepatitis, the method comprises administrating the extracts from Morus alba L. or the main active ingredients thereof to the subject.

21. The method according to claim 14, wherein the extracts from Morus alba L. are extracts from Ramulus Mori, Cortex Mori and/or Folium Mori.

22. The method according to claim 15, wherein the non-alcoholic fatty liver disease comprises any one or more of steatosis, non-alcoholic steatohepatitis, hepatic fibrosis, and hepatic cirrhosis.

23. The method according to claim 15, wherein,
a method for preparing the extracts from Morus alba L. comprises:
1) preparing crude extract solution from Moraceae plants;
2) subjecting the crude extract solution to cation resin and/or optional anion resin for separation to obtain the extracts from Morus alba L.

24. The method according to claim 23, wherein the method further comprises:
3) subjecting a resin effluent from step 2) to alcohol precipitation and collecting the supernatant;
4) concentrating and drying the supernatant;
alternatively, the method further comprises a step of concentrating and drying the resin effluent from step 2).

25. The method according to claim 14, wherein the main active ingredients of the extracts from Morus alba L. comprises at least one of 1-deoxynojirimycin, N-methyl-1-deoxynojirimycin, fagomine, 3-epi-fagomine, 1,4-dideoxy-1,4-imino-D-arabinitol, calystegine B2, calystegine C1, 2-O-(α-D-galactopyranosyl)-1-deoxynojirimycin, 6-O-(β-D-glucopyranosyl)-1-deoxynojirimycin, and 1,4-dideoxy-1,4-imino-(2-O-β-D-glucopyranosyl)-D-arabinitol.

26. The method according to claim 14, wherein the subject comprises a mammal, preferably, the mammal is human.

27. The method according to claim 14, wherein the extract from Morus alba L. or the main active ingredient thereof is administrated orally.
